SEQUENCE LISTING (16 sequences)

SEQ ID NO: 1 - DNA, 16 bp, Homo sapiens
agcctagcttagactt

SEQ ID NO: 2 - DNA, 15 bp, Homo sapiens
gcctagcttagactt

SEQ ID NO: 3 - DNA, 14 bp, Homo sapiens
cctagcttagactt

SEQ ID NO: 4 - DNA, 13 bp, Homo sapiens
cctagcttagctt

SEQ ID NO: 5 - DNA, 13 bp, Homo sapiens
tagcttagactta

SEQ ID NO: 6 - DNA, 13 bp, Homo sapiens
agcttagacttag

SEQ ID NO: 7 - DNA, 13 bp, Homo sapiens
gcttagacttagc

SEQ ID NO: 8 - DNA, 13 bp, Homo sapiens
gcttagacttagc

SEQ ID NO: 9 - DNA, 13 bp, Homo sapiens
ttagacttagcct

SEQ ID NO: 10 - DNA, 13 bp, Homo sapiens
tagacttagccta

SEQ ID NO: 11 - DNA, 13 bp, Homo sapiens
agacttagcctag

SEQ ID NO: 12 - DNA, 13 bp, Homo sapiens
gacttagcctagc

SEQ ID NO: 13 - DNA, 13 bp, Homo sapiens
acttagcctagct

SEQ ID NO: 14 - DNA, 13 bp, Homo sapiens
cttagcctagctt

SEQ ID NO: 15 - DNA, 14 bp, Homo sapiens
ttagcctagcctta

SEQ ID NO: 16 - DNA, 13 bp, Homo sapiens
tagcctagcttag

(19) United States
(12) Patent Application Publication
(10) Pub. No.: US 2007/0037146 A1
(43) Pub. Date: Feb. 15, 2007

(54) Biodisc microarray and its fabrication, use, and scanning

(76) Inventor: Gilbert H. Hong, Los Altos Hills, CA (US)

Correspondence Address: VIERRA MAGEN MARCUS & DENIRO LLP, 575 MARKET STREET SUITE 2500, SAN FRANCISCO, CA, 94105, US

(21) Appl. No.: 10/412,973

(22) Filed: Apr. 14, 2003

(51) Int. Cl. C12Q 1/68; C12M 1/34

(52) U.S. Cl. 435/6; 435/287.2

(57) ABSTRACT

A biodisc comprises a CD-type optical disc with small feature oligonucleotide probes disposed on its surfaces. The biodisc's probes are either custom-fabricated in-situ with a master-duplicate tandem arrangement of discs that allows one disc and a reading/tracking head to control the probe locations being synthesized pass-by-pass on the duplicate. Or the biodisc is mass-produced in a manufacturing process that includes a spin-on-and-peel (SOAP) method to create a nickel stamper for standardized biodisc oligonucleotide probes. Such biodisc is fabricated with four masks only, and these are shifted between depositions to synthesize particular individual 4-mer+ target nucleotide probes.

### BACKGROUND OF THE INVENTION

1. Field of the Invention

The present invention relates to microarrays of oligonucleotide probes, and more particularly to the fabrication, use, and reading of microarrays disposed on compact disc type substrates that are rotationally scannable in optical media computer disc drives.

2. Description of Related Art

DNA microarrays are glass micro slides or nylon membranes with genomic DNA, cDNA, oligonucleotides, or other DNA samples in ordered two-dimensional matrices. Biochip DNA microarrays use oligonucleotides and measure the length of each probe's sequences in “mers”. Typical biochip oligonucleotides probes are 40, 50, 60, and 70 mers in length. Recent publications on the ideal length of oligonucleotide probes, as well as experimental evidence and development by MWG Biotech AG research indicates that 50-mer oligonucleotide probes show an optimal balance of sensitivity and specificity.

DNA microarrays are used to analyze gene expression and genomic clones or to detect mutations, single nucleotide polymorphisms (SNPs). The microarray DNA selected is often from a group of related genes such as those expressed in a particular tissue, during a certain developmental stage, in certain pathways, or after treatment with drugs or other agents. Expression of that group of genes is quantified by measuring the hybridization of fluorescent-labeled RNA or DNA to the microarray-linked DNA sequences. Transcriptional changes can be monitored by gene expression profiling through organ and tissue development, microbiological infection, and tumor formation.

DNA samples are now being automatically analyzed with so-called DNA microarray chips. Such semiconductor chips are able to sense where fluorescent-dye marked DNA fragments have attached themselves to cells arranged on the chip. Each cell has an address and a fragment of a DNA double helix. Samples with unknown fragments of DNA double helix material are floated by the chip surface. These will attach themselves, in a natural DNA-recombination process called hybridization, at various cell addresses of the DNA microarray chip. The DNA sequences of the sample material fragments will be revealed by chip addresses where each fragment attaches itself. The sample material fragments are therefore prepared with fluorescent dye to later make their address-of-attachment optically visible by the chip.

There are a very large number of genetic sequences possible. Far too many to fit on a single microarray. So conventional practice is to engineer microarrays that have those genetic sequences that are likely to be useful in a particular line of research. Such microarrays are either purchased as standard models from a catalog, or created in-situ as the need arises.

A prior art DNA microarray chip of this sort is described by Paola Arena, et al., in United States Patent Application US2002/0097900 A1, published 7/25/2002. Such uses VLSI CMOS semiconductor technology to fabricate the chip. A binary on/off indication is obtained at each pixel of an image frame processed by the chip.

Agilent uses its inkjet technology to print oligos and whole cDNAs onto glass slides. The non-contact inkjet technology produces microarrays with more uniform and consistent features. The inkjet technology is used as part of an industrial-scale manufacturing process that enables us to reliably produce uniform, high quality microarrays from lot to lot. Since the inkjet process is a non-contact process, it does not introduce defects as a result of surface tension interactions with the microarray surface. Thus, Agilent's non-contact inkjet process provides substantial improvement over pin spotting with respect to consistency and spot uniformity. The number of features depends on the type of microarray. Up to 16,200 features can be microarrayed on Agilent's cDNA catalog microarrays. Of these, there are a number of control features, and orientation markers. The Human-1 cDNA Microarray includes 13,675 individual, microarrayed clones in addition to a series control genes and orientation markers. For Agilent's custom in-situ oligonucleotide microarrays, there are two formats. From 8,400 to 22,000 individual oligonucleotides can be microarrayed in these two formats.

Biologically relevant Deoxyribose nucleic acids (DNA) basically consists of four bases, adenine (A), guanine (G), cytosine (C) and thymine (T). A given sequence of bases such as ATTGCATGA will bind its complementary strand TAACGTACT to form a stable duplex. Thus biological information is stored in a one dimensional sequence of bases.

Sequencing by hybridization (SbH) is a sequence analysis technology that exploits the natural base pairing to decode the nitrogenous bases of assays of interest. Prior art devices attach synthetic DNA base sequences to substrates to form “probes” which will hybridize to complementary base sequences in a sample “target” DNA fragment. See Mitchell D. Eggers, et al., “Genosensors, micro fabricated devices for automated DNA sequence analysis,” SPIE Vol. 1891, Advances in DNA Sequencing Technology (1993), pp. 113-126. Probes can be made in different lengths with different numbers of bases. For example, all of 65,536 different 8-base probes will be needed to detect all the possible complementary 8-base sequences that can occur anywhere in a target sample. The particular 8-base probes that hybridize the target sample will tell which particular 8-base sequences occur. Target samples longer than 8-bases can be completely read by combining 8-base probes according to their overlapping patterns. For example, a probe match of two 8-base sequences, ATTTCGGA and TTTCGGAG, can indicate a 9-base sequence in a target of ATTTCGGAG.

A particular oligonucleotide probe array system is marketed by Affymetrix (Santa Clara, Calif.) under the trademark GENECHIP. Various oligonucleotide patterns are arranged on the probe in engineered sequences. Hewlett-Packard (Palo Alto, Calif.) makes a scanner for the GENECHIP system that can read the fluorescent glows from target fragments that bind at the surface of the probes.

Special application software then interprets what nucleic acid sequences were present in the target from both the X, Y location of the light that was read from the probe and the light's relative intensity. The typical probe is organized as a flat two-dimensional array with X, Y addresses. Target nucleic acids are fluorescently labeled for hybridization to ligands with X, Y addresses in the probe arrays. See, Peter M. Goodwin, et al., “DNA sequencing by single-molecule detection of labeled nucleotides sequentially cleaved from a single strand of DNA,” SPIE Vol. 1891, Advances in DNA Sequencing Technology 5 (1993), pp. 127-131.

Prior art inkjet technology provides a very flexible and relatively inexpensive way to fabricate chip-type microarrays. But the feature density is not high, and the reading of such microarrays requires expensive scanners. Prior art disc-format microarrays achieve better densities, but are limited in being able to resolve small probe locations.

### SUMMARY OF THE INVENTION

An object of the present invention is to provide a system for reading nucleic acid sequences in biological assays of interest.

Another object of the present invention is to provide a microarray with a relatively large oligonucleotide probe capacity.

A further object of the present invention is to provide a microarray with a relatively dense oligonucleotide probe organization.

A still further object of the present invention is to provide a CD-format microarray that can be scanned with inexpensive CD-type optical disc drives.

Briefly, a biodisc embodiment of the present invention comprises a CD-type optical disc with small feature oligonucleotide probes disposed on its surfaces. The biodisc's probes can be custom-fabricated in-situ with a master-duplicate tandem arrangement of discs that allows one disc and a reading/tracking head to control the probe locations being synthesized pass-by-pass on the duplicate. The biodisc can also be mass-produced in a manufacturing process that includes a spin-on-and-peel (SOAP) method to create a nickel stamper for standardized biodisc oligonucleotide probes. Such biodisc is fabricated with four masks only, and these are shifted between depositions to synthesize particular 4-mer+ target nucleotide probes.

An advantage of the present invention is that a biodisc is provided that has a dense, high population of oligonucleotide probes.

A further advantage of the present invention is that a mass production method is provided for making of an oligomer master.

Another advantage of the present invention is that a mass production method is provided for replicating of oligomer discs for DNA hybridization and assay.

A further advantage of the present invention is that a simple method and device are provided for DNA hybridization and assay.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIGS. 1A and 1B diagram a biodisc system embodiment of the present invention, and FIG. 1B illustrates the vertical arrangement of oligonucleotide probes, not to scale, that are disposed along a spiral track on the surface of the biodisc;

FIG. 2 is a flowchart of a method for sequencing nucleic acid according to the present invention;

FIG. 3 is a cross-section of a 16-base biodisc embodiment of the present invention that uses only four masks in its fabrication;

FIG. 4 is a functional block diagram of an in-situ probe synthesizing system embodiment of the present invention;

FIG. 5 represents a process for making optical-disc oligomer-microarray masters;

FIG. 6 represents a method embodiment of the present invention for making biodiscs with a SOAP-conformal image transfer method; and

FIG. 7 represents a transparent, flat hollow diskette in a floppy disc type case with a slider door, and in which microarrays of oligonucleotide probes are disposed on the inside surfaces of the hollow, and so that samples can wash by, hybridize and their attachments be optically viewable from outside through the slider door.

### DETAILED DESCRIPTION OF THE INVENTION

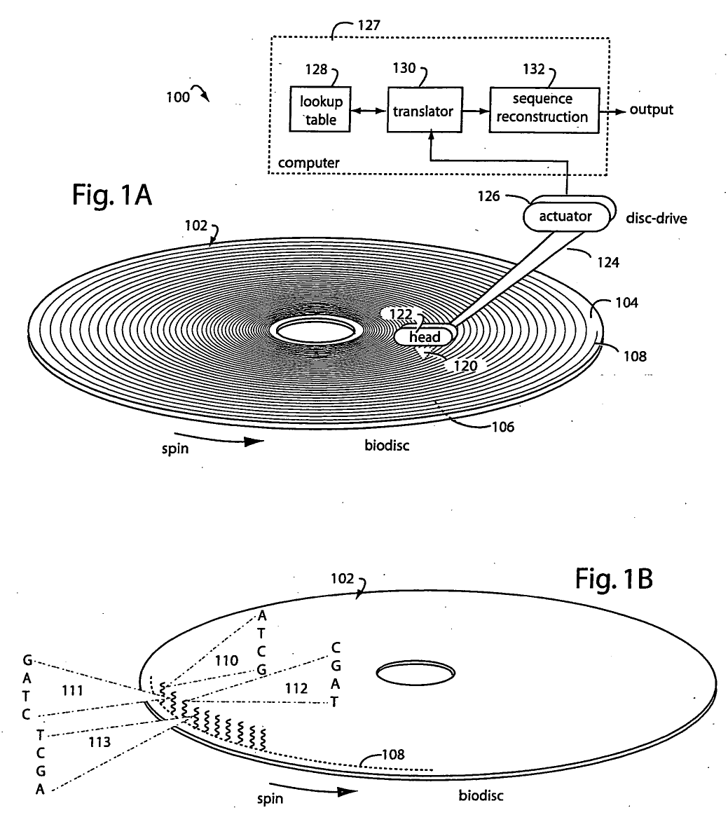

FIG. 1A illustrates an biodisc for sequencing by hybridization (SbH) in a system embodiment of the present invention, referred to herein by the reference numeral 100. The SbH system 100 includes a flat disc substrate (biodisc) 102 that resembles a compact disc (CD). Such substrate format affords many advantages in that relatively inexpensive and readily available CD mastering, duplication, and optical player technology and equipment can be used. The biodisc 102 includes opposite surfaces 104 and 106. A spiral 108 is disposed on one surface and comprises a one-dimensional array of sites with unique physical addresses. For example, the physical addresses can be referenced by the relative angle (theta) and radius (R), or by a sequential sector or block number. Preferred embodiments of the present invention use a physical addressing scheme that is similar to those used for audio-CD and CD-ROM discs so that the corresponding disc-player technology can be adapted.

A biodisc system includes a biodisc with a variety of oligonucleotide probes with different sequences disposed on its surfaces at predetermined and addressable locations. A disc drive provides for mounting and optical scanning of the biodisc. A computer is connected to the disc drive and has a-priori information about the predetermined and addressable locations of the oligonucleotide probes disposed on the biodisc. During scanning, its software is able to detect any hybridization of particular ones of the oligonucleotide probes when the biodisc is mounted on the disc drive with the help of flouroluminescent tags on the samples. Thus the particular genetic sequences in a sample material that were in contact with the biodisc can be identified to a researcher.

FIG. 1B illustrates four oligonucleotide probes 110-113 that are typical of those disposed along spiral 108. For sake of example, these are illustrated with a 4-base vertical depth (i.e., 4-mer). Probe lengths of 20-70 mer are more typical in actual practice. The illustrations of FIG. 1B represent these probes as being corkscrew, and that physical structure is a natural occurrence in these sorts of molecules.

A synthetic-DNA probe 110 has a first physical address and comprises a base sequence, for example, of ATCG, reading from outside-to-substrate. A synthetic-DNA probe 111 has a second physical address and comprises another base sequence, for example, GATC. A synthetic-DNA probe 112 has a third address and comprises a base sequence, for example, CGAT. A synthetic-DNA probe 113 has a fourth physical address and comprises, for example, a base sequence of TCGA.

FIG. 1B merely shows one example, and practical embodiments of the present invention will have as many as a billion unique synthetic-DNA probes each with as much as a 20-base depth, e.g., giving 4 to the 20^{th} power unique sequences (roughly 1 trillion) that can be matched.

Returning to FIG. 1A, after hybridization of a target, a light beam 120 is used by a combination light source and detector head 122 to read any light returned from hybridized nucleotide probes on the spiral 108. A motor and spindle are used to spin the biodisc 102 as in a conventional CD-type optical disc drive. An actuator 124 connects to a servo motor 126 such that the head can be positioned at any radial position relative to the biodisc 102. A computer 127 provides scanning and analysis functions. An inventory of the oligonucleotide probes is cataloged in a database according to their physical addresses on the biodisc 102. A lookup table 128 provides an index into this catalog that is used by a translator 130 to convert physical addresses on the spiral to the corresponding base-sequence in a reconstruction processor 132.

In some embodiments of the present invention, it may be advantageous to optically encode computer data directly on the biodisc 102 using CD-ROM techniques that includes the information for lookup table 128. For example, to avoid errors matching the physical synthetic-DNA addresses to the base-sequence information from the hybridization.

The DNA oligos arrays can be arranged in parallel with digitally encoded CD-tracks for servo tracking, probe markings, alignment, labeling, coding schemes, and other information. Such encodings can be used to identify the synthesized genes, the number of probes, the address location within the biodisc, part numbers, manufacturer names, etc.

In embodiments of the present invention, the overall surface area can be very large, e.g., as in a ten-inch diameter standard video-disc. Such large surface areas can be used to advantage to provide enough space for a billion or more unique synthetic-DNA probes. These probes can mix, e.g., 6-base, 20-base, 64-base and even 250-base synthetic-DNA probes. Fluorescent markers attached to a target sample will provide tell-tales as to how much and exactly which probes were hybridized.

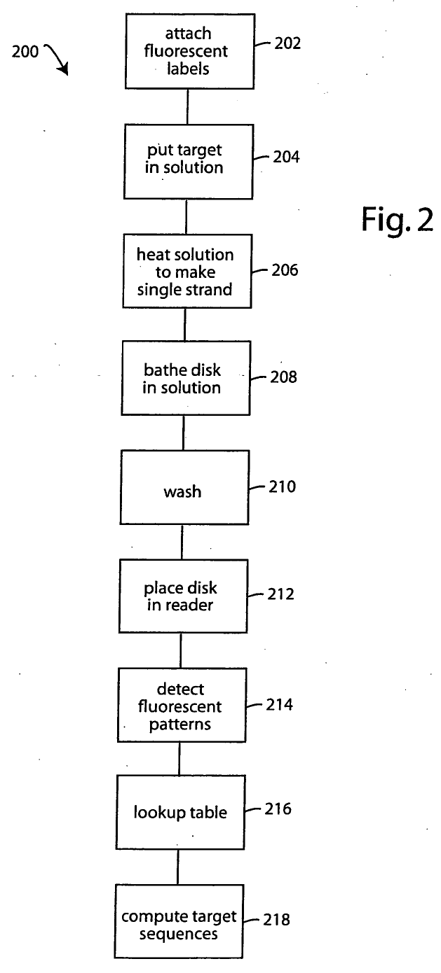

FIG. 2 represents a nucleic acid sequencing method embodiment of the present invention, and is referred to by the general reference numeral 200. In a step 202, fluorescent-labeled receptors are attached to a target sample, or a synthetic-DNA probe, or both. The fluorescent label can be attached covalently or by intercalation, and may comprise ethidium bromide, succinylfluoresceins, FITC, NBD, Texas Red, and tetramethylrhodamine isothiocynate. The covalent attachment can be done either chemically or enzymatically. The typical illumination source will be ultraviolet (UV), e.g., 308 nanometers (nm).Luminescence will usually be detected in the red spectrum, e.g., 630 nm. A step 204 places a target sample in solution. A step 206 heats the solution to obtain single-strand target material. A step 208 bathes a disc, such as biodisc 102 in FIGS. 1A and 1B, in the solution for hybridization. A step 210 washes away any unreacted solution.

A step 212 places the disc in a disc-player apparatus such as is shown as system 100 in FIG. 1A. A step 214 illuminates the disc in ultraviolet light and detects any red spectrum fluorescence, e.g., disc sector block-by-block until the entire longitudinal length of spiral 108 is read. A lookup table is referenced in a step 216 that translates the physical addresses that show some fluorescence into the corresponding nucleic acid sequences for the synthetic DNA-probes located at those sites. A step 218 computes the target sequences by interpolating the data from step 216. Overlaps in different sequences are used to string together sequences with bases larger in number than the disc has.

In a biodisc fabrication process, e.g., in a 16-base CD-format that is 130 mm by 15 mm, as many as eight billion synthetic-DNA probes can be accommodated with spot sizes as small as 0.8 micron and both sides of the biodisc are used.

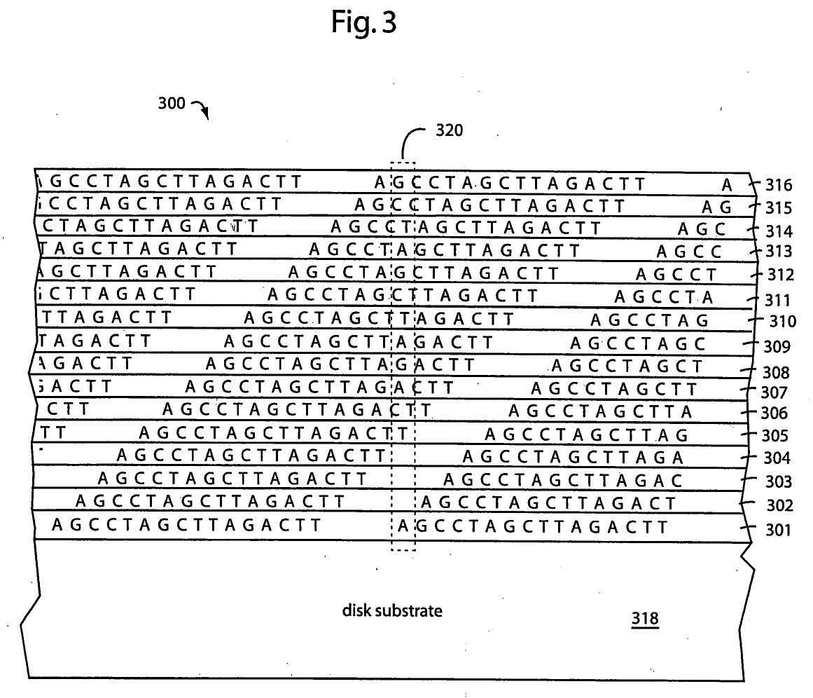

Referring to FIG. 3, a first layer 301 is deposited on the substrate of the biodisc that repeats a oligomer base pattern, e.g., “AGCCTAGCTTAGACTT”. This pattern is deposited along the entire length of the spiral using four oligomer deposition cycles that each follow photoresist and masking, e.g., “A”, “G”, “C”, and “T”. The design of each such mask and their combination provides the information needed to preload the lookup table 128 (FIG. 1A).

The depositions eventually build a single continuous ridge that is grooved in between adjacent rings. Such fabrication technology uses semiconductor industry photoresist and exposure masks. See, U.S. Pat. No. 5,468,324, issued Nov. 21, 1995, to the present inventor, Gilbert H. Hong.

Referring again to FIG. 3, a second layer 302 repeats the pattern in the first layer 301, but shifts the oligomer base pattern “AGCCTAGCTTAGACTT” forward by one. This overlaying and shifting forward by one is repeated for every successive layer 303 through 316. Such technique pattern shifting lowers manufacturing costs because only four masters are needed.

These masters are preferably used sixteen times to get 16-mers, twenty times for 20-mer, etc. A typical synthetic-DNA probe 320 has a “GCCTAGCTTAGACTTA” sequence. FIG. 3 shows two identical sequences. One analysis showed that total number of sequences that can be built this way is the total gross probe capacity of the biodisc divided by sixteen. Only one sequence is functional, the remaining fifteen are only partially hybridizable. A saw-tooth intensity distribution graph a leading edge up to address sixteen and drop off sharply at seventeen, and climb back up to a second peak at thirty-two. At which point, hybridization is complete and stable again.

So, millions of probes can be accommodated on a biodisc by using FOUR masks repeated sixteen times. The total number of available sequences is the total number of bits (pits) divided by sixteen.

A target material in solution must be able to make actual contact with the nucleotide probe so hybridization can occur. Any stray disc substrate or adjacent probe material can block access to a probe. In alternative embodiments of the present invention, the synthetic-DNA probes are each fabricated as independent free-standing stacks that are aligned in a row.

Each layer in a biodisc can be produced with four exposure and depositions cycles, e.g., one for each of A, G, T, and C. For example, at an arbitrary starting address of “1”, a first layer has a “AGCCTAGCTTAGACTT” pattern deposited. Table I summarizes the four mask patterns that are needed to do this.

TABLE I

| layer-1 | A | G | C | C | T | A | G | C | T | T | A | G | A | C | T | T |
|---|---|---|---|---|---|---|---|---|---|---|---|---|---|---|---|---|
| Mask-A | x | | | | | x | | | | | x | | x | | | |
| Mask-G | | x | | | | | x | | | | | x | | | | |
| Mask-C | | | x | x | | | | x | | | | | | x | | |
| Mask-T | | | | | x | | | | x | x | | | | | x | x |

Table II shows the pattern for layer-2 relative to address “1”. This pattern is easily implemented by moving the substrate one position to the left. Compare Tables I and TABLE II.

TABLE II

| layer-2 | T | A | G | C | C | T | A | G | C | T | T | A | G | A | C | T | T |
|---|---|---|---|---|---|---|---|---|---|---|---|---|---|---|---|---|---|
| Mask-A | | x | | | | | x | | | | | x | | x | | | |
| Mask-G | | | x | | | | | x | | | | | x | | | | |
| Mask-C | | | | x | x | | | | x | | | | | | x | | |
| Mask-T | x | | | | | x | | | | x | x | | | | | x | x |

Table III shows the pattern for layer-3 relative to address “1”. This pattern is also easily implemented by advancing the substrate (disc) one more step to the left. Compare Tables II and III.

TABLE III

| layer-3 | T | T | A | G | C | C | T | A | G | C | T | T | A | G | A | C | T | T |
|---|---|---|---|---|---|---|---|---|---|---|---|---|---|---|---|---|---|---|
| Mask-A | | | x | | | | | x | | | | | x | | x | | | |
| Mask-G | | | | x | | | | | x | | | | | x | | | | |
| Mask-C | | | | | x | x | | | | x | | | | | | x | | |
| Mask-T | x | x | | | | | x | | | | x | x | | | | | x | x |

In summary, four-mask patterning and shifting method embodiments of the present invention greatly reduce the number of total masks required in biodisc manufacturing. A standard CD has an recordable area of around one hundred square centimeters. If four square microns are needed per address, two million such addresses will fit on a standard CD sized substrate. With sixteen addresses per usable probe, one hundred million usable probes can be realized with only four stampers or masks, e.g., one for each base. Biodisc embodiments of the present invention can use conventional constant angular velocity (CAV) or constant linear velocity (CLV) recording formats, as well as concentric and spiral track arrangements. The spiral track format is slightly preferred in biodisc 102.

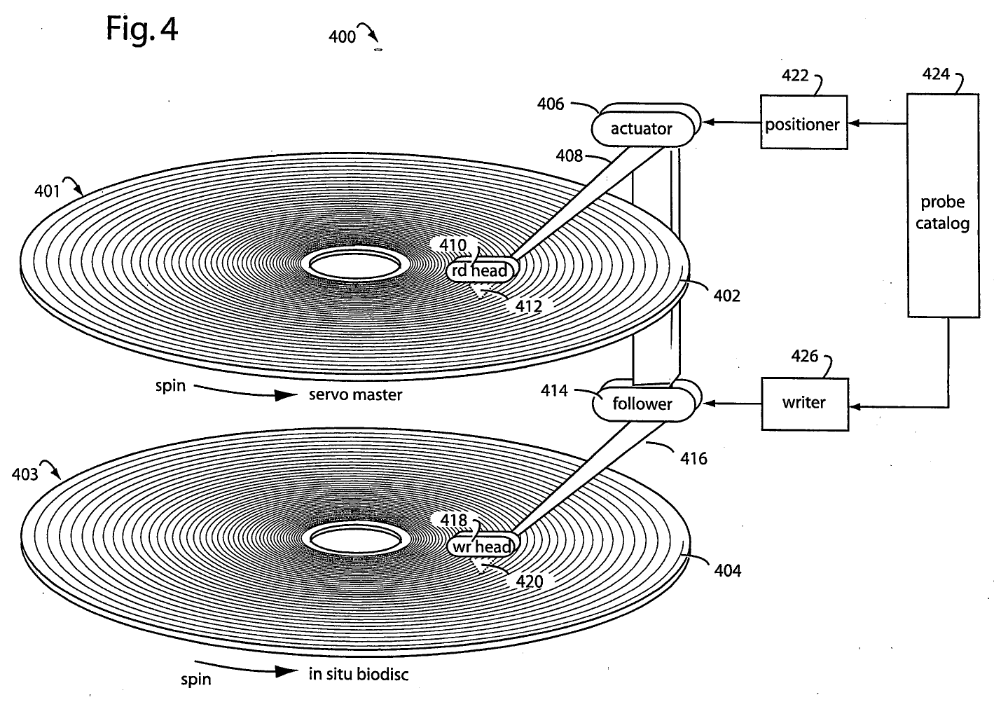

FIG. 4 represents an in-situ probe synthesizing system 400. A highly repeatable addressing of the probes on a biodisc being fabricated is needed, so the in-situ probe synthesizing system 400 includes a servo master disc 401 with an address index spiral 402. These parallel an in-situ biodisc 403 that has a oligonucleotide probe spiral 404 that is to be synthesized. Both discs spin together on the same drive spindle. The in-situ biodisc 403 is demountable. An actuator 406 moves a servo tracking arm 408 and read head 410. A light beam 412 is used to track the address index spiral 402. A follower 414 exactly tracks actuator 406 with a tracking arm 416 and write head 418. A laser 420 writes patterns on biodisc 403 for later deposition of the four base materials, ATCG. A positioner 422 converts the electronic commands from a probe catalog 424 into disc addresses. The probe catalog 424 maps the inventory of oligonucleotide probes needed to be written onto biodisc 403 by their assigned physical addresses. Such database of catalog information is later used by scanners to interpret the results of hybridization. A writer 426 electronically drives write head 418 according to which base material is needed at any one spot, probe by-probe.

It is also possible to etch deep into the underlying glass substrates and make the necessary tracks and channels. Such tracks can be etched deep enough to accommodate all 20-mer of a probe inside each pit along the track channel.

System 400 is used for in-situ synthesis of oligomers on a biodisc. The servo master disc 401 provides for highly repeatable addressing of new biodisc 403 on which the in-situ oligomers being synthesized. The read/tracking head 410 associated with the servo master disc 401 is mechanically paired with recording head 418 associated with the new biodisc 403. For example, the heads share a common armature. Such arrangement allows the recording head to revisit the same locations on the new biodisc with very high accuracy and repeatability. This is necessary to be able to correctly build up the nucleotide probe sequences that need separate recording exposure passes for each A, T, C, and G component. Alternatively, the disc can be a two-sided configuration with servo/alignment tracks on one side for the read/tracking head and the synthesized oligomers on the other side accessible to the recording head. In one embodiment, the new biodisc is demountable and the servo master disc is not. Such would yield a production uniformity during manufacturing. A Sony WMC3300, or other advanced laser beam recorder can be used with some modification to meet any special requirements. For example, an alignment mark added to the biodisc substrate and special software and hardware for multiple exposures.

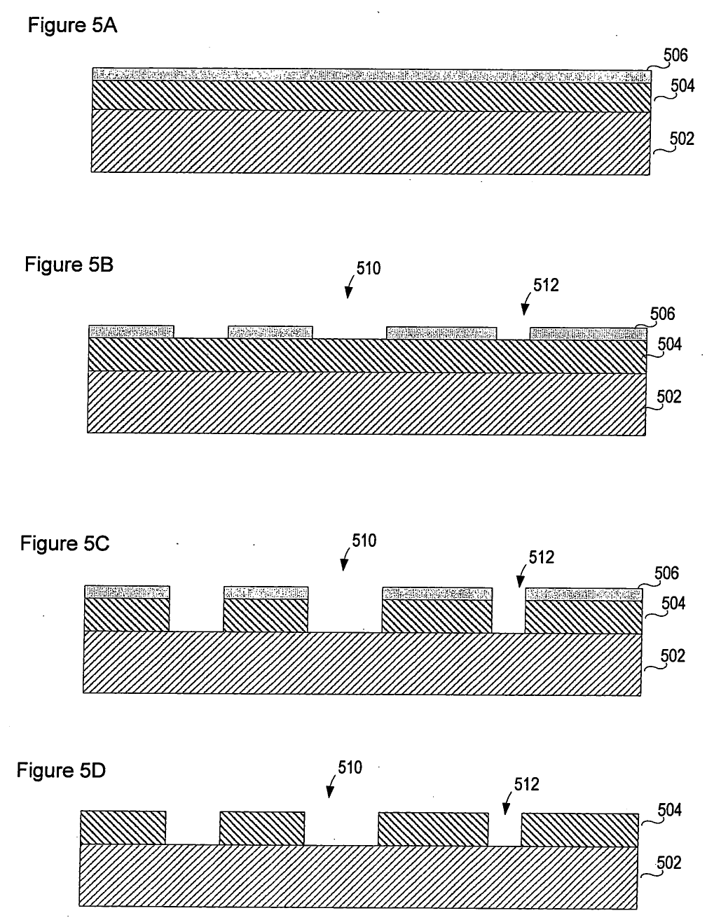

FIG. 5 represents a process for making optical-disc oligomer-microarray masters. In a step 502, a circular glass disc with chromium plating is coated with photoresist and placed in a laser beam recorder with a rotating spindle chuck. In a step 504, the recording uses laser beams modulated by piezo-electric or acoustic methods to expose the photoresist. The photoresist images are developed after laser exposure. In a step 506, the patterns formed by the remaining photoresist are etched through the chrome layer. The resist is stripped in a step 508, and the remaining patterned chrome becomes a stamper that matches the original image written by the LBR. Duplicates can then be mass produced using these chrome masters, e.g., as in conventional semiconductor and compact disc manufacturing processes.

Duplicate biodiscs can be made with conventional projection printing with no reduction. This is a 1:1 process and masters can be photomasks or stampers. Duplicates can also be made using a step-and-repeat process, e.g., 5× or 10×.

A method embodiment of the present invention can be used to make duplicates with a combination of the SOAP and conformal image transfer processes. Such is similar to 1× contact process used in making sub-masters from masters. No reduction is possible, but small features can be successfully replicated.

In another embodiment of the invention, a release layer is deposited in between the glue layer and the master. In which case, the release material will be transferred to the substrate. It is also possible to have a material that will stay with the master and not be transferred and is preferable because the release layer can be reused over and over again. For example, thin sputtered gold layer is inert and non-sticking.

An alternative method embodiment of the present invention uses SOAP-conformal image transfer in making biodiscs. The image from master is replicated and used as flood exposure masks. The protected area will not be subject to chemical addition but the unprotected area will become active for another addition. Alternatively, the image can be used as a stencil for adding another base. The chemical addition is done at uncovered area. After stripping the first SOAP-conformal image transfer layer, the process is repeated and chemical addition at the unprotected area can be done. The SOAP layer is a chemical mask, instead of a photo mask. Results are similar, the differentiation is based on optical and chemical and photochemical properties of the SOAP-conformal image transfer layers.

Data tracks and bio-tracks can be placed side by side. The data, DNA probe track (bio-track) can be interdigitated in one singe complete spiral tracks, (2) the data track and biotrack can be divided into two spirals and not mixed, and (3) disc area can be divided into separate data track and biotrack areas. All these are permissible and offer the highest degree of flexible, depending on design and usages. CD-formats are quite flexible, audio, video, data can be presented in one disc. The biotracks can be commingled with other CD-audio, CD-data, and DVD standard tracks.

Circular masters, such as stampers, are preferably made using a laser beam recorder exposure system. The desired pattern is recorded by modulating the laser beam. The laser exposes patterns on the photoresist-coated glass substrate. Subsequent development of the latent image, etching, and electroforming is used to transfer the pattern to a hard nickel stamper. The resist is coated with thin silver layer, placed in a nickel tank and a nickel thin layer is grown over the resist image. The nickel stamper is removed and the resist is stripped. The stamper can be used thousands, and even millions times to produce duplicates that resemble regular compact discs.

In alternative method embodiments, the glass substrate can be coated with both a metal chromium layer and a photo-sensitive photoresist layer. A laser beam recorder expose tool patterns the resist layer. The photoresist image was developed first. The developed resist image is then used as a mask for the etching of the chromium layer. Finally, resist is stripped, we have a hard surface chromium mask.

A photochemical in-situ synthesis of DNA oligos is marketed by Affymetrix, Inc. Two distinct exposure methods can be used, flood exposure with masks to define desired exposure patterns, or step-by-step along the track one address at a time with laser beam recording.

The circular-format biodisc duplicates can be manufactured using conventional semiconductor and compact disc techniques. Once a master is in-hand, conventional photolithographic equipment can be used to make duplicates. Full size 1:1 projection and step-and-repeat reduction methods can be used for biodisc embodiments of the present invention. How to use photolithographic and light directed probe synthesis is straightforward. These are non-contact methods of making biodiscs.

The word contact only refers to the close proximity between the master and its duplicate. In the usual sense, contact printing means the master and duplicate are in actual contact and a vacuum is applied to remove any gap between. But the uneven surfaces cause the contact to be less than perfect, so errors result. Method embodiments of the present invention are different in this respect, they use a near perfect contact in which gaps between the master and its duplicate do not exist.

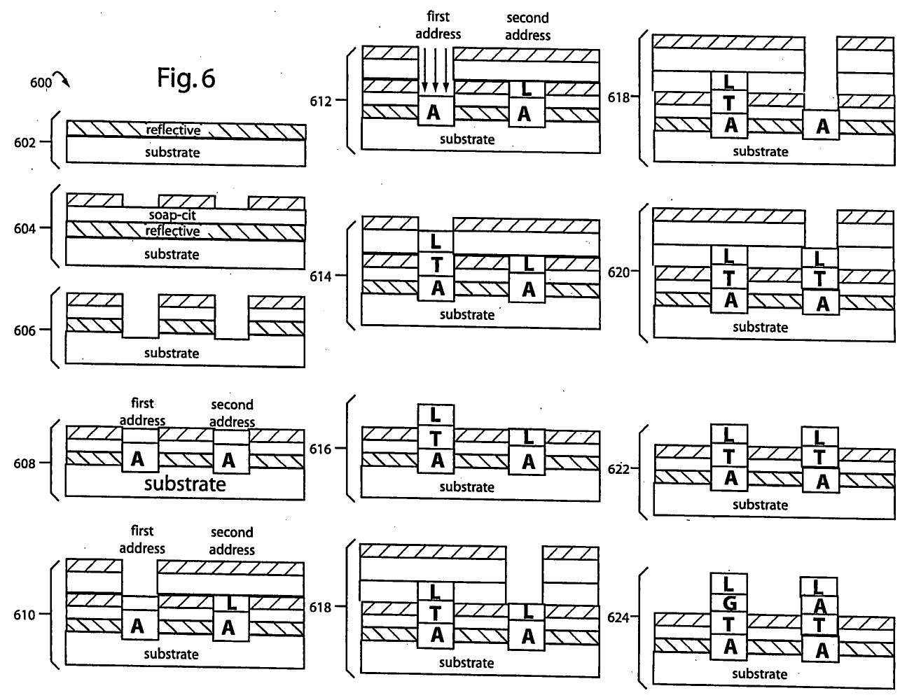

FIG. 6 represents a method embodiment of the present invention for making biodiscs with a SOAP-conformal image transfer method, and is referred to herein by the general reference numeral 600. In a step 602, a reflective chrome layer is deposited on a glass substrate. In alternative embodiments, a light-absorbing layer is used to replace the chrome so that a dark background will be provided to help contrast fluorescent-dye marked hybridization sites of the oligonucleotide probes being formed.

A spin-on-and-peel (SOAP) conformal image transfer (CIT) layer is placed to define lands and pits, in a step 604. A step 606 etches away portions of the reflector and some glass to open up pits. A base, e.g., “A”, is deposited in the pits and capped with a protective layer in a step 608. Conformal image transfer and flood exposure are used in a step 610 to remove the protective layer, e.g., at a first address only. LBR is not used. In a step 612, another base, “T”, is deposited on the first address with chemistry. In a step 614, the CIT layer is stripped. Another CIT layer is deposited in a step 616 to expose the second address and simultaneously protect the first address. In a step 618, the protective layer is removed at the second address using flood exposure. In a step 620, a base, “G”, is added to the second address. In a step 622, the CIT layer is stripped. At this point, the pits are aligned along a track and interdigitated with groove channels. A land area straddles both sides of the along-track string of grooves and pits, and runs parallel. Such land areas separate adjacent tracks from one another. The oligonucleotide probes are thus being constructed in the pits and are separated from each other along-track by the groove areas. A step 624 represents the repetition of the previous steps in building additional layers.

The image from a master is replicated and used as a flood exposure mask. The protected area will not be subject to chemical addition but the unprotected area will become active for another addition. In another variation of the invention, the image can be used as a stencil for adding another base. The chemical addition is done at uncovered area. After stripping the first SOAP-conformal image transfer layer, the process is repeated and chemical addition at the unprotected area can be done. In this usage, the SOAP layer is a chemical mask, instead of a photo mask. Results are similar, the differentiation is based on optical and chemical and photochemical properties of the SOAP-conformal image transfer layers. Certain material will be used as a chemical mask whereas others might be used as a photo mask. Compared to conventional photolithographic method, the current method does not require the use of photo-sensitive material and also eliminating the conventional developing step. More flexibility and wider choice of material and process is a distinct advantage of the present invention.

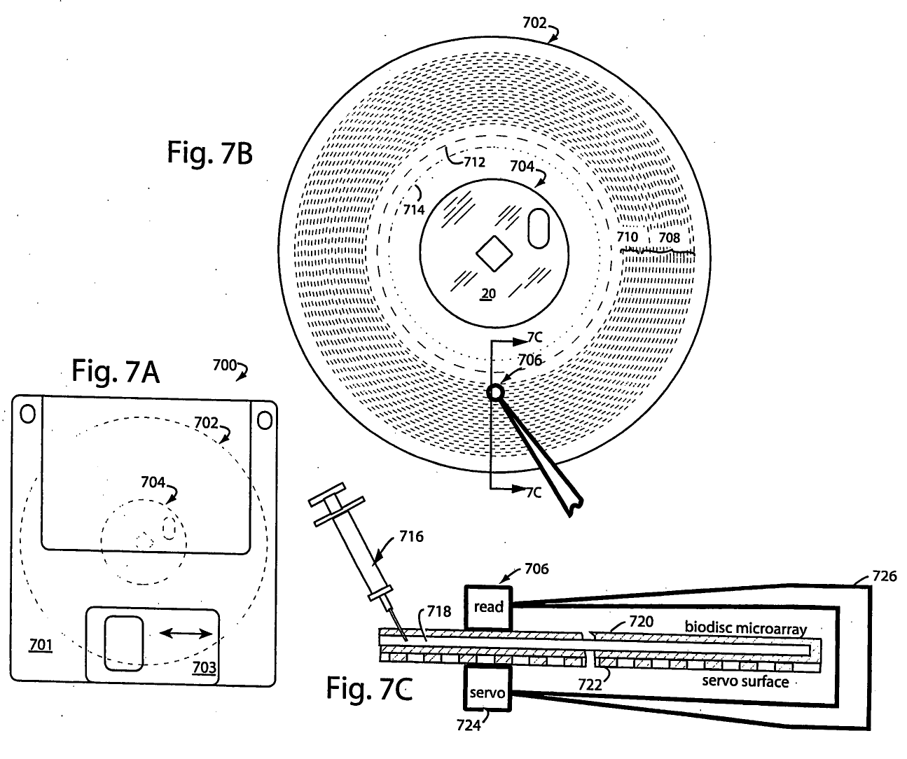

FIG. 7 represents a transparent, flat hollow diskette in a floppy disc type case with a slider door, and in which microarrays of oligonucleotide probes are disposed on the inside surfaces of the hollow, and so that samples can wash by, hybridize and their attachments be optically viewable from outside through the slider door.

FIG. 14 A typical design for circular biodiscs. Notice that the possibility of spinning the disc not only make it easier to read and write but also might help in hybridization. The chemical reaction can be accelerated with spinning also.

Although particular embodiments of the present invention have been described and illustrated, such is not intended to limit the invention. Modifications and changes will no doubt become apparent to those skilled in the art, and it is intended that the invention only be limited by the scope of the appended claims.

The invention claimed is:

1. A biodisc, comprising:
a round (plastic, metal or glass) disc having a concentric middle hole or hub for a spindle and opposite flat planar sides; and
a number of oligonucleotide (or protein or other biological interesting polymers)probes with different sequences disposed along tracks at known locations on at least one of said opposite flat planar sides.

2. The biodisc of claim 1, further comprising:
a number of servo tracks on a first or second of said opposite flat planar sides and providing for repeatability in addressing of individual ones of the number of oligonucleotide probes disposed on a first or second one of said opposite flat planar sides.

3. A biodisc system, comprising:
a biodisc with a variety of oligonucleotide (or protein or other biopolymers) probes with different sequences disposed on its surfaces at predetermined and addressable locations;
a disc drive for mounting and optical scanning of the biodisc; and
a computer connected to the disc drive and having a-priori information about said predetermined and addressable locations of said oligonucleotide probes disposed on the biodisc, and able to detect any hybridization of particular ones of said oligonucleotide probes when the biodisc is mounted on the disc drive, and further able to thereby identify particular genetic sequences in a sample material that was in contact with the biodisc.

4. An method for manufacturing a in-situ synthesized biodisc, comprising:
coupling together a servo master disc and an in-situ biodisc together on a common spindle;
paralleling a read head for tracking position information on said servo master disc, and a writing head for recording oligonucleotide information on said in-situ biodisc together on a common actuator;
determining a particular oligonucleotide for synthesis at a location on the in-situ biodisc;
addressing each location on said in-situ biodisc indirectly with said read head for tracking position information on said servo master disc;
depending on said common spindle and said common actuator to repeatably address said locations on said in-situ biodisc as said particular oligonucleotide is in the process of being synthesized.

5. A mass-production method for manufacturing a biodisc, comprising:
using only four different masks in shifted angular positions during each of a plurality of masking operations to synthesize oligonucleotide sequences on a biodisc that exceed four-mer.

6. An biodisc, comprising:
a flat planar disc substrate;
a spiral disposed on at least one surface of the disc substrate;
a first oligomer deposited at a first physical address in the spiral and having an affinity during hybridization for a first DNA particle; and
a second oligomer deposited at a second physical address in the spiral and having an affinity during hybridization for a second DNA particle.

7. The biodisc of claim 6, wherein:
the first and second oligomers and the first and second physical addresses occur in a particular sequence.

8. The biodisc of claim 6, further comprising:
a first DNA particle hybridized to the first oligomer and that has been dyed with a fluorescent; and
a second DNA particle hybridized to the second oligomer and that has been dyed with a fluorescent.

9. The biodisc of claim 8, wherein:
the first and second physical addresses occur in a particular sequence and a fluorescent glow from such location is evidence of a hybridization of a DNA fragment with a first DNA particle in sequence with a second DNA particle.

10. A nucleic acid sequencing method, comprising the steps of:
attaching fluorescent-labeled receptors to a target sample or a synthetic-DNA probe, or both;
placing said target sample in a solution;
heating said solution to obtain a single-strand target material;
hybridizing a biodisc in said solution;
washing away any unreacted solution;
reading patterns of hybridization as indicated by said fluorescent-labeled receptors present at particular physical addresses while rotating said biodisc, and scanning radially across with a detector head until an entire longitudinal length of a synthetic-DNA spiral on a surface of said biodisc is read;
using a lookup table to translates a detected physical address into a complementary nucleic acid sequence for a corresponding synthetic DNA-probe; and
computing a target sequence by interpolating data obtained by from said lookup table.

11. A method for fabricating a biodisc that has a spiral line of synthetic-DNA probes, the method comprising the steps of:
depositing a first layer on a biodisc substrate that repeats a oligomer base pattern along an entire length of the spiral line using four oligomer deposition cycles “A”, “G”, “C”, and “T”;
depositing a second layer that repeats said oligomer base pattern on top of said spiral line and said first layer, but shifts the oligomer base pattern forward by one probe; and
depositing n-number of layers that repeat said oligomer base pattern on top of said spiral line and a lower layer, but shifts the oligomer base pattern forward by one;
wherein, at least 4ⁿ uniquely sequenced synthetic-DNA probes each with n-base sequences are fabricated on said substrate for hybridization of nucleic acid target samples.

12. A biodisc for hybridization of nucleic acid target samples, comprising:
a flat, round disc substrate for optical scanning in a CD-type disc drive;
a plurality of independent and individually unique synthetic-DNA probes deposited on the disc substrate in a single layer comprising A, T, C, and G affinity oligomers;
wherein, each said synthetic-DNA probe is laid down on its side.

13. The biodisc of claim 12, wherein:
plurality of independent and unique synthetic-DNA probes have at least two subclasses of with different synthetic-DNA probes numbered base sequences.

14. The biodisc of claim 12, wherein:
the flat disc substrate has an outside diameter of at least ten inches; and
the plurality of independent and unique synthetic-DNA probes are deposited in four different masking cycles, one for each of A, T, C, and G.
